(19) United States
(12) Patent Application Publication
(10) Pub. No.: US 2023/0181829 A1
(43) Pub. Date: Jun. 15, 2023

(54) INJECTION APPARATUS AND METHOD FOR AQUATIC SPECIES

(71) Applicant: pHi-Tech Animal Health Technologies Ltd., Airport City (IL)

(72) Inventors: Gershon Goldenberg, Pardes Hanna-Karkur (IL); Asaf Halamish, Pardes Hanna-Karkur (IL); Nethanel Bab, Pardes Hanna (IL); Assaf Shafran, Jerusalem (IL)

(73) Assignee: pHi-Tech Animal Health Technologies Ltd., Airport City (IL)

(21) Appl. No.: 18/107,437

(22) Filed: Feb. 8, 2023

Related U.S. Application Data
(63) Continuation of application No. PCT/IB2021/057450, filed on Aug. 12, 2021.
(60) Provisional application No. 63/066,026, filed on Aug. 14, 2020.

(51) Int. Cl. A61M 5/20; A61M 5/142; A61M 5/168; A61M 5/315

(52) U.S. Cl. CPC A61M 5/204; A61M 5/14216; A61M 5/16831; A61M 5/31546; A61M 5/31551

(57) ABSTRACT

An injection apparatus can include a hand-held unit having a head portion and a body portion. The head portion can include a base member, a needle fluidly coupled to the base member, a movable portion disposed over the needle and being axially movable relative to the base member between an extended position and a retracted position in which a distal point of the needle is exposed, a safety cover removably coupled to the base member, the safety cover comprising one or more extension members, and an adjustment member removably coupled to the movable portion and configured to adjust a penetration depth of the needle by increasing or decreasing a distance between the movable portion and the base member.

### CROSS-REFERENCE TO RELATED APPLICATIONS

The present application is a continuation of International Application No. PCT/IB2021/057450, filed Aug. 12, 2021, which claims the benefit of U.S. Provisional Application No. 63/066,026, filed on Aug. 14, 2020, both of which applications are incorporated herein by reference in their entireties.

### FIELD

The present disclosure concerns injection apparatuses for injecting subjects, particularly aquatic species including fish, or feed or companion animals (e.g., poultry, swine, cattle, sheep, goats, ungulates, cats, and dogs) with one or more medicaments.

### BACKGROUND

In the aquaculture industry, aquatic stock (e.g., aquatic organisms such as fish, crustaceans, mollusks, algae, aquatic plants, marine animals, and other organisms) often must be administered substances such as medications for a variety of reasons. Producers must treat large numbers of aquatic stock, and treatment can require injecting each aquatic organism with a plurality of medications, generally in liquid form. Such medications can include medicines, vaccines, hormones, food supplements and the like (hereinafter referred to generally as “medicament”). Administering such medicaments typically includes using an administration device, such as a syringe or drenching unit, from which a medicament dose is either manually or automatically administered to the animal. This type of administration generally includes hand actuation or pumping of the device to deliver medicament to the animal.

Treating a large number of aquatic organisms (e.g., via a hand-actuated device) causes operator fatigue, which can result in a number of malfunctions including, for example: (i) accidental self-injection by the operator; (ii) administering the medicament dose when the needle has not yet penetrated the subject's skin or has not yet penetrated to the desired depth; (iii) administering the dose after the needle has been removed from the subject; (iv) administering only a portion of the required dose; (v) inserting the needle into the subject in a non-optimal orientation; and/or (vi) administering a duplicate dose to the same individual, among others.

Additionally, in some cases, it is necessary to administer more than one medicament to a single aquatic organism. In cases where there is no ready composition of the different medicaments, or where it is not possible to combine two or more medicaments together, more than one injection must be performed per subject, which again contributes to operator fatigue.

Accordingly, there is a continuing need for improved injector devices and methods for their use, such as devices for injecting a large number of aquatic species with one or more medicaments.

### SUMMARY

Described herein are embodiments of an injection apparatus for administering one or more medicaments to a subject, particularly an aquatic species, such as fish or marine animals. The injection apparatus can be used to administer medicament to a large number of subjects in a quick and efficient manner to prevent and/or reduce operator error, operator fatigue, and injection malfunction.

In a representative embodiment, an injection apparatus can comprise a hand-held unit having a head portion and a body portion. The head portion comprises a needle, a support portion, and a movable portion. The movable portion is axially movable relative to the support portion between an extended position and a retracted position in which a distal point of the needle is exposed. The injection apparatus can further comprise a safety cover removably coupled to the movable portion. Certain embodiments of the safety cover comprise a base member and a lockplate pivotably coupled to the base member. The lockplate is pivotable between a locked position in which the safety cover and movable portion are restrained from axial movement relative to the needle and an unlocked position in which the safety cover and movable portion can move axially relative to the needle.

Certain embodiments of the safety cover can further comprise first and second clip members that extend from the base member and are configured such that positioning a subject between the clip members aligns a selected injection site on the subject with the distal point of the needle. The clip members can be movable between an open position and a gripping position in which the clip members are configured to grip the subject.

The safety cover can further comprise a biasing member configured to bias the lockplate into the locked position.

The injection apparatus can further comprise a needle support portion configured to releasably couple the needle to the body portion. For these embodiments, the lockplate comprises a locking member that extends from the lockplate and has a locking surface configured to engage a corresponding locking surface on the needle support portion when the lockplate is in the locked position.

The safety cover can further comprise an annular protrusion having a threaded outer surface configured to couple a correspondingly threaded surface of a connector coupled to the movable portion.

The lockplate can further comprise an aperture extending through a thickness of the lockplate. The aperture can align with the needle such that when the movable portion is in the retracted position at least a portion of the needle extends through the aperture. The injection apparatus can further comprise a removal member disposed within the aperture. The removal member comprises a needle aperture extending through a thickness of the removal member. The needle aperture is sized such that an inner surface of the removal member contacts an outer surface of the needle to remove contaminants from the outer surface of the needle.

The injection apparatus can further comprise at least one control unit remote from the hand-held unit and fluidly coupled to the hand-held unit via one or more connecting tubes. The control unit may comprise a pump. In some embodiments, the control unit is configured to be worn by a user.

The hand-held unit can be configured to be submersible in water.

The movable portion can further comprise a lock mechanism movable between a locked position, wherein the movable portion is restrained from moving into the retracted position, and an unlocked position, wherein the movable portion can move between the extended position and the retracted position. The lock mechanism can be configured for actuation by any convenient method, such as actuated by a trigger coupled to the body portion.

The injection apparatus can be configured to operate in and switch between at least an automatic mode and a manual mode. When the apparatus is in automatic mode, medicament can be administered automatically upon insertion of the needle to a selected depth within a subject. When the apparatus is in manual mode, medicament is administered when an operator actuates a trigger coupled to the body portion.

In another representative embodiment, an injection apparatus can comprise a base station comprising a base plate, an upper member coupled to the base plate, and head portion removably coupled to the upper member. The head portion comprises a needle, a support portion, and a movable portion. The movable portion is axially movable relative to the support portion between an extended position and a retracted position in which a distal point of the needle is exposed. Disclosed embodiments of the apparatus may further comprise a safety cover removably coupled to the upper member. The safety cover comprises a base member and a lockplate pivotably coupled to the base member. The lockplate is pivotable between a locked position, in which the safety cover and movable portion are restrained from axial movement relative to the needle, and an unlocked position, in which the safety cover and movable portion can move axially relative to the needle.

The injection apparatus may comprise at least one control unit remote from the base station and fluidly coupled to the upper member via one or more connecting tubes, the at least one control unit comprising a pump. The upper member can be configured to communicate with the control unit via a communicating cable.

The base station and the safety cover can be submersible in water.

The safety cover can further comprise a biasing member configured to bias the lockplate into the locked position. The injection apparatus can further comprise a needle support portion configured to releasably couple the needle to the body portion. The lockplate may comprise a locking member that extends from the lockplate and has a locking surface configured to engage a corresponding locking surface on the needle support portion when the lockplate is in the locked position.

The present invention also includes a method comprising providing disclosed embodiments of the injection apparatus and using the apparatus to administer medicament to a subject. In a representative embodiment, a method of using an injection apparatus can comprise urging the lockplate against a selected implantation site on a subject thereby pivoting the lockplate from the locked position to the unlocked position, actuating the movable portion to move the movable portion and the safety cover relative to the needle to expose the distal point of the needle, and administering a medicament when the distal point of the needle has reached a selected depth within the subject. The method can further comprise first and second clip members extending from the base member such that a selected injection site on the subject is aligned with the distal point of the needle.

The movable portion can further comprise a lock mechanism movable between a locked position wherein the movable portion is restrained from moving into the retracted position and an unlocked position wherein the movable portion can move between the extended position and the retracted position. Accordingly, the method may further comprise actuating a trigger operatively coupled to the lock mechanism to move the lock mechanism to the unlocked position.

In another representative embodiment, a method can comprise: providing an injection apparatus comprising a head portion having a needle; a support portion; a movable portion axially movable relative to the support portion between an extended position and a retracted position in which a distal point of the needle is exposed; and a safety cover removably coupled to the movable portion, the safety cover comprising a base member and a lockplate pivotably coupled to the base member, the lockplate being pivotable between a locked position in which the safety cover and movable portion are restrained from axial movement relative to the needle and an unlocked position in which the safety cover and movable portion can move axially relative to the needle. The method can further comprise urging the lockplate against a selected injection site on a subject such that the lockplate pivots into the unlocked position. The movable head is actuated to insert the distal point of the needle into the subject. A medicament is administered when a distal point of the needle has reached a selected depth within the subject.

The method can further comprise positioning a selected injection site of a subject between first and second clip members extending from the base member to align the selected injection site with the distal point of the needle. The method can further comprise gripping the subject using the first and second clip members.

The movable portion can further comprise a lock mechanism movable between a locked position wherein the movable portion is restrained from moving into the retracted position and an unlocked position wherein the movable portion can move between the extended position and the retracted position, and the method can further comprise actuating a trigger operatively coupled to the lock mechanism to move the lock mechanism to the unlocked position.

In some embodiments, the head portion is coupled to a body portion to form a hand-held unit configured to be carried by an operator. In other embodiments, the head portion is coupled to a base station comprising a base plate and an upper member coupled to the base plate, the base station configured to be coupled to a surface.

In a representative embodiment, an injection apparatus can comprise a hand-held unit having a head portion and a body portion. The head portion can comprise a base member, a needle fluidly coupled to the base member, a movable portion disposed over the needle and being axially movable relative to the base member between an extended position and a retracted position in which a distal point of the needle is exposed, a safety cover removably coupled to the base member, the safety cover comprising one or more extension members, and an adjustment member removably coupled to the movable portion and configured to adjust a penetration depth of the needle by increasing or decreasing a distance between the movable portion and the base member.

### BRIEF DESCRIPTION OF THE DRAWINGS

FIG. 1 is a perspective view of an exemplary injection apparatus.

FIG. 2 is a perspective view of the head portion of the injection apparatus of FIG. 1.

FIG. 3 is a cross-sectional perspective view of the head portion of the injection apparatus of FIG. 1.

FIG. 4 is an exploded view of the hand-held unit of the injection apparatus of FIG. 1, with the safety cover removed.

FIG. 5 is a perspective view of the head portion of the injection apparatus of FIG. 1 holding an exemplary subject.

FIG. 6 is a cross-sectional view of a portion of the head portion of FIG. 2, shown in the extended and locked position.

FIG. 7 is a cross-sectional view of a portion of the head portion of FIG. 2, shown in the retracted and unlocked position.

FIG. 8 is a perspective view of an exemplary head portion for an injection apparatus.

FIG. 9 is a perspective view of an exemplary removal member.

FIG. 10 is a top plan view of an exemplary safety cover for an injection apparatus, shown in the gripping position.

FIG. 11 is a top plan view of the safety cover of FIG. 10, shown in the open position.

FIG. 12 is a side view of an operator wearing an exemplary injection apparatus.

FIG. 13 is a perspective view of an exemplary injection apparatus.

FIGS. 14-15 are perspective views of an exemplary head portion for an injection apparatus.

FIG. 16 is an exploded perspective view of the head portion of FIG. 14.

FIGS. 17-18 are perspective views of the base member of the head portion of FIG. 14.

FIG. 19 is a perspective view of the support member of the head portion of FIG. 14.

FIGS. 20-21 are perspective view of the adjustment member of the head portion of FIG. 14.

FIG. 22 is a perspective view of the movable portion of the head portion of FIG. 14.

FIG. 23 is a perspective view of the safety cover of the head portion of FIG. 14.

FIG. 24 is a perspective view of the head portion of FIG. 14.

FIG. 25 is a perspective view of the head portion of FIG. 14 with the movable portion removed.

FIG. 26 is a perspective view of the head portion of FIG. 14 with the movable portion shown in the retracted configuration.

FIG. 27 is a perspective view of the head portion of FIG. 14 including another exemplary embodiment of a safety cover.

FIG. 28 is a side elevation view of the head portion of FIG. 14.

FIG. 29 is a cross-sectional view of the head portion of FIG. 28 taken along line A-A.

FIG. 30 is a side elevation view of the head portion of FIG. 14.

FIG. 31 is a cross-sectional view of the head portion of FIG. 30 taken along line B-B.

### DETAILED DESCRIPTION

### I. Definitions

Certain aspects, advantages, and novel features of disclosed embodiments are described herein. Disclosed embodiments of the apparatus, system and method should not be construed as being limiting in any way. Instead, the present disclosure concerns all novel and nonobvious features and aspects of the various disclosed embodiments, alone and in various combinations and sub-combinations with one another. The apparatus, system and method are not limited to any specific aspect or feature or combination thereof, nor do the disclosed embodiments require that any one or more specific advantages be present, or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily understood by a person of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the operator and further away from the administration site. As used herein, the term “distal” refers to a position, direction, or portion of a device that is further away from the operator and closer to the administration site. Thus, for example, proximal motion of a device is motion of the device away from the administration site and toward the operator (e.g., away from the subject's body), while distal motion of the device is motion of the device away from the operator and toward the administration site (e.g., into the subject's body). The terms “longitudinal” and “axial” refer to an axis extending in the proximal and distal directions, unless otherwise expressly defined.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited. As used herein, the term “about” means the listed value still produces the functional result associated with the value, and may be within 10% of the listed value. For example, “about 100 degrees” means at least any value between 90-110 degrees, inclusive.

The term “medicament” as used herein refers to any substance that may be administered to a subject. Particular embodiments include, for example and without limitation, antibiotics, vaccines, therapeutics, hormones, food supplements, oils, vitamins, minerals, etc. In some embodiments, the medicaments are in liquid form. In other embodiments, the medicaments may be in powdered form and may be mixed with one or more liquids or solvents to form a suspension or solution within the two or more containers or prior to being disposed therein. Particular exemplary medicaments include, but are not limited to: PAQVAC® STREP INIAE, PAQVAQ® STREP AGALACTIAE, KV3®, ALPHA JECT micro® 1 Noda, ALPHA JECT®, 2000, Alpha ERM Salar, ALPHA JECT micro® 7 IL, ALPHA JECT micro® 6, ICHTIOVAC®VNN, AQUAVAC® Irido V, AQUAVAC® Strep SA, and combinations thereof.

The term “subject” as used herein refers to a human or an animal subject to a treatment, observation, or experiment.

The term “animal” can refer to an aquatic animal, a land animal, an avian, or an amphibian. For example, animals can include but are not limited to: poultry, swine, cattle, sheep, goats, horses, deer, felines, canines, and/or aquatic species including fish. Cattle may be a dairy animals or animals raised for beef. Animals can include animals raised for human consumption or a domesticated animal. Examples of animals that can be injected using the disclosed injection apparatus embodiments include, but are not limited to, ruminant species, such as a sheep, goat, cow, heifer, bull, bullock, calf, ox, deer, bison, buffalo, elk, alpaca, camel or llama; ungulates, such as a horse, donkey, or pig; avians, such as chickens, including laying hens and broilers, turkey, goose, duck, Cornish game hen, quail, partridge, pheasant, guinea-fowl, ostrich, emu, swan, or pigeon; aquatic animals, such as an aquaculture species, such as fish (e.g., salmon, trout, tilapia, sea bream, carp, cod, halibut, snapper, herring, catfish, flounder, hake, smelt, anchovy, lingcod, moi, perch, orange roughy, bass, tuna, mahi, mackerel, eel, barracuda, marlin, Atlantic ocean perch, Nile perch, Arctic char, haddock, hoki, Alaskan Pollock, turbot, freshwater drum, walleye, skate, sturgeon, Dover sole, common sole, wolfish, sablefish, American shad, John Dory, grouper, monkfish, pompano, lake whitefish, tilefish, wahoo, cusk, bowfin, kingklip, opah, mako shark, swordfish, cobia, croaker, or hybrids thereof, and the like), crustaceans (e.g., lobster, shrimp, prawns, crab, krill, crayfish, barnacles, copepods, and the like), or mollusks (e.g., squid, octopus, abalone, conchs, rock snails, whelk, clams, oysters, mussels, cockles, and the like). Additionally, or alternatively, the animal may be a companion animal, such as canines; felines; rabbits; rodents, such as a rat, mouse, hamster, gerbil, guinea pig or chinchilla; birds, such as parrots, canaries, parakeets, finches, cockatoos, macaws, parakeets or cockatiel; reptiles, such as snakes, lizards, tortoises or turtles; fish; crustaceans; and amphibians, such as frogs, toads and newts.

The terms “food supplement,” “dietary supplement,” and “feed additive” as used herein can refer to products intended to supplement the diet of a subject. Food supplements can include, but are not limited to, vitamins, fatty acids, probiotics, minerals, amino acids, enzymes, herbs and botanicals (including plant materials, algae, macroscopic fungi, and combinations thereof), and other substances.

### II. Exemplary Embodiments

Disclosed herein are embodiments of an injection apparatus useable to administer one or more medicaments to a subject (for example, aquatic species including fish), either simultaneously or sequentially. Though the below embodiments of the injection apparatus are described with respect to their use on aquatic species such as fish, it should be understood that the embodiments disclosed herein can be used on any of a variety of subjects.

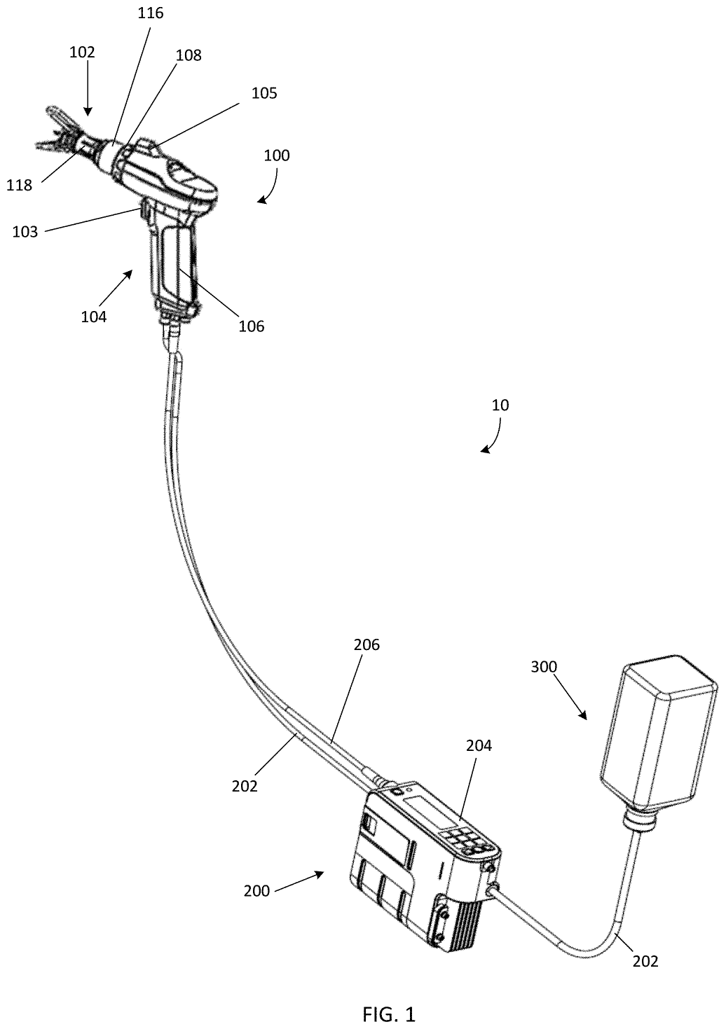

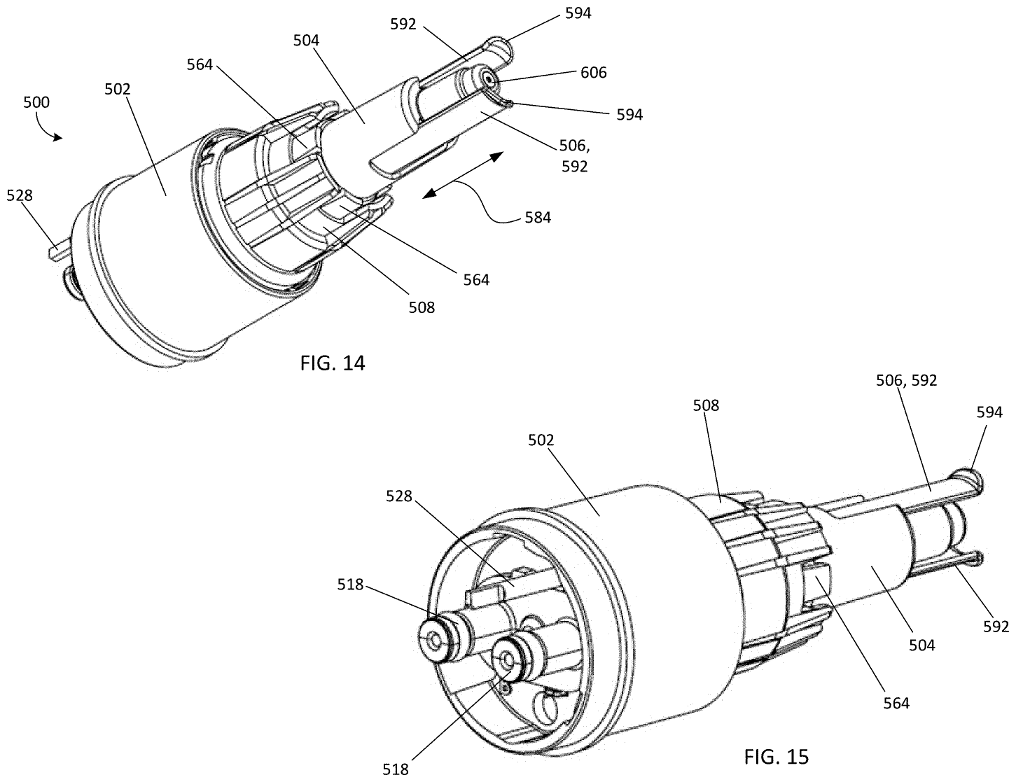

FIGS. 1-7 illustrate an exemplary embodiment of an injection apparatus 10 including a hand-held unit 100 comprising an interchangeable head portion 102 (or head portion 500 shown in FIGS. 14-20) removably coupled to a body portion 104. In certain embodiments, injection apparatus 10 is useful for injecting one or more medicaments into a subject. Referring to FIG. 1, a disclosed injection apparatus embodiment 10 can comprise a hand-held unit 100, at least one control unit 200 fluidly coupled to the hand-held unit by at least one connecting tube 202, and one or more medicament containers 300 removably coupled to the at least one control unit 200 and/or to the hand-held unit 100 by one or more connecting tubes 202. In other embodiments, the containers 300 can be coupled to the hand-held unit 100 and/or the control unit 200 directly.

The injection apparatus 10 can be a modular apparatus wherein each of the components (e.g., hand-held unit 100, head portion 102 and/or 500, body portion 104, control unit 200, containers 300) is interchangeable and/or replaceable. For example, the injection apparatus 10 can be provided as an assembly or kit and can be packaged together for delivery to the end user. An exemplary kit can include a body portion 104, one or more control units 200, one or more containers 300, flexible tubing 202, and one or more head portions 102 and/or 500. In some embodiments, each provided head portion 102 and/or 500 can be configured particularly for use with a different type of subject (e.g., aquatic species, poultry, swine, etc.). Details of additional head portions for use with poultry, swine, and/or other subjects can be found, at least, in International Application No. PCT/IB2020/053177 and International Publication No. WO 2019/116229, each of which is incorporated herein by reference in its entirety. In other embodiments, each kit can include several head portions 102 such that the head portion 102 can be replaced if it becomes damaged and/or worn.

Replacement or additional components (e.g., additional head portions 102, 500 and/or additional pumps for the control unit 200) can also be provided separately from the kit in order to replace components of the modular injection apparatus 10. Such a modular configuration advantageously allows the injection apparatus 10 to be adapted for a variety of uses, including for use with a variety of subjects. The modular configuration further allows an operator to switch between head portions 102, 500 (and therefore between needle types, e.g., between needles having differing lengths and/or widths, between subdermal or subcutaneous needles, between stationary and movable needles, etc.), and/or replace damaged needles or head portions, for example, in a field or operation setting. Such a configuration allows an operator to mitigate disruption to the injection process caused by a broken needle or a damaged and/or blocked head portion. In some embodiments, the control unit 200 can further comprise a replaceable pump that can be removed and replaced without the use of tools.

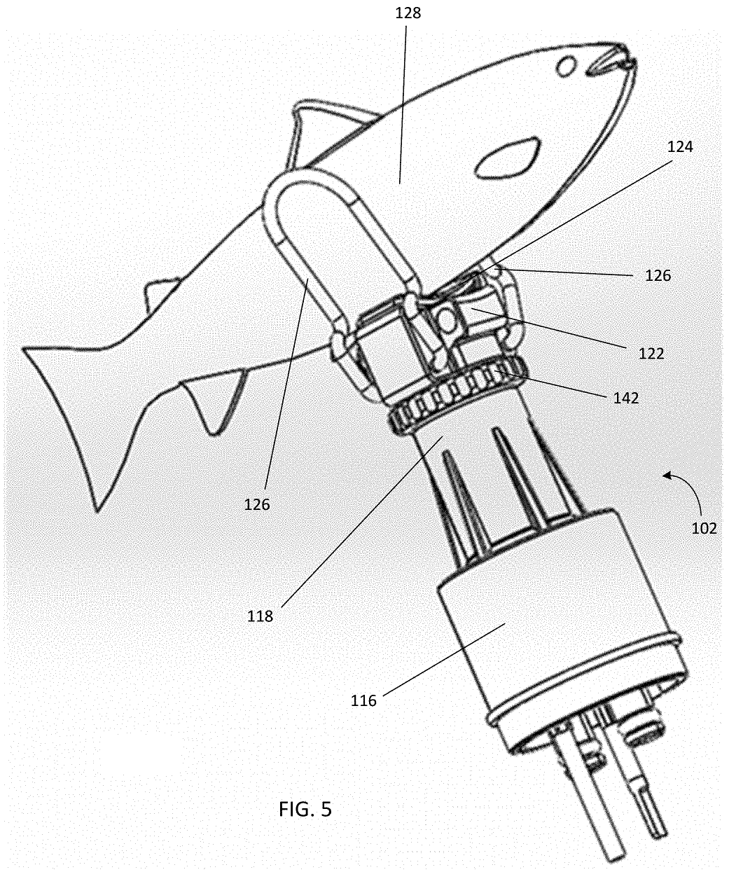

As mentioned, the hand-held unit 100 can comprise an interchangeable head portion 102 and/or 500 that can be removably coupled to the body portion 104. As shown in FIG. 5, the head portion 102, 500 can be configured for use with subjects such as aquatic species including fish. The body portion 104 can include a gripping portion 106 to be held by an operator. The hand-held unit 100 can further comprise a light 105 coupled to, for example, the body portion 104.

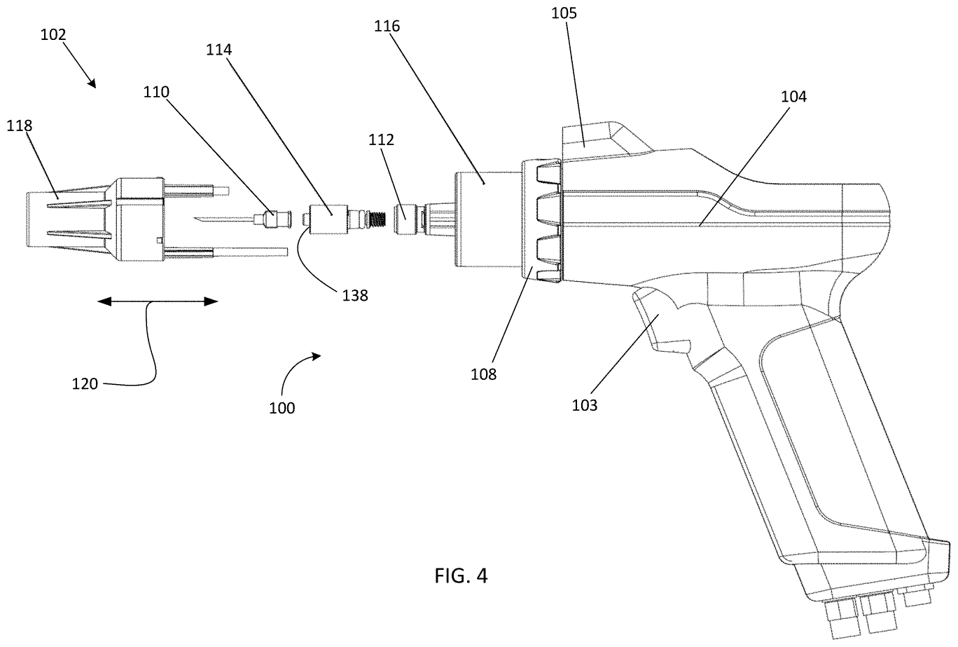

The head portion (e.g., head portion 102 and/or 500) can comprise a connector 108 (FIG. 4) configured to couple a corresponding connector of the body portion 104. For example, the connector 108 can have an internal threaded surface configured to couple an external threaded surface of the body portion, or vice versa. To remove and/or replace the head portion 102, 500, the connector 108 can be rotated in a first direction (e.g., counterclockwise) causing the threads of the connector 108 to disengage from the body portion 104, thus causing the head portion 102, 500 to disengage from the body portion 104. The connector 108 can be rotated in a second direction (e.g., clockwise) to couple the head portion 102 (or a replacement head portion or alternate head portion) to the body portion 104. In other embodiments, the head portion 102 can be coupled to the body portion 104 using, for example, any suitable connector or connecting system, such as a snap-fit connection, clips, or other mechanical means such as screws etc.

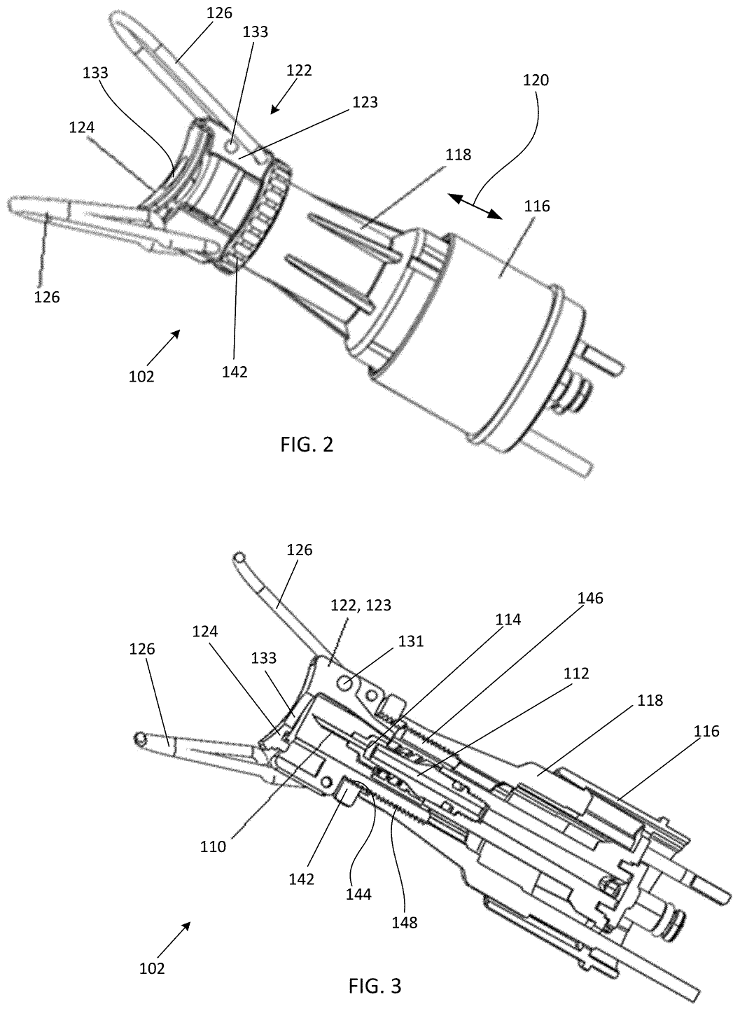

Referring to FIGS. 2-3, the head portion 102 can comprise one or more needles 110 configured to administer medicament to the subject. In the illustrated embodiment, as shown in FIG. 3, the head portion 102 comprises one needle 110; however, in other embodiments, the head portion 102 can comprise two or more needles. FIG. 4 shows an exploded view of the hand-held unit 100 with safety cover 122 removed for purposes of illustration. Referring to FIG. 4, the needle 110 can be fluidly coupled to a connecting tube 112 via a needle support portion 114 comprising, for example, a luer lock configured to releasably couple the needle 110. The connecting tube 112 can be fluidly coupled to the body portion 104 and to the containers 300 via the control unit 200.

The head portion 102 can comprise a support portion 116 and a movable portion 118. The movable portion 118 can be axially movable relative to the support portion 116 in a telescoping manner, as shown by arrow 120. The movable head 118 can be movable between an extended position (see e.g., FIG. 3) wherein the distal point (e.g., distal edge) of the needle is covered by the safety cover 122 and a retracted position (see e.g., FIG. 7) wherein the distal point of the needle 110 is exposed. In some embodiments, the movable head 118 can comprise a biasing member (e.g., a spring, a compressible sleeve, etc.) configured to bias the movable head into the extended position. The needle support portion 114 (and therefor the needle 110) can be coupled to the body portion 104 and/or the support portion 118 such that the movable portion 118 can move relative to the needle 110.

Urging the movable portion 118 rearwardly (e.g., proximally) relative to the support portion 116 exposes a distal point of the needle 110 and allows the needle to be inserted into the subject's body. In some embodiments, urging the movable portion 118 rearwardly into the retracted position (e.g., by pressing the movable portion 118 against a selected injection location on the subject) triggers the release of a predetermined dose of medicament (e.g., by opening a valve associated with the needle and/or by triggering movement of a pump within the control unit 200). The movable portion 118 can further comprise a lock mechanism configured to, when engaged, prevent the movable portion 118 from moving relative to the support portion 116. The lock mechanism can be operatively coupled to the trigger 103 (FIG. 4), such that actuating (e.g., pressing) the trigger 103 disengages the lock mechanism and allows the movable portion 118 to move relative to the support portion 116. Further details of the movable portion and support portion can be found, for example, in International Application No. PCT/IB2020/053177

Referring again to FIGS. 2-3, the needle 110 can be covered by a safety cover 122, which is removably coupled to the movable portion 118 (e.g., via a connector 142). The safety cover 122 can be configured to selectively cover the needle 110 and can comprise a base 123, a lockplate 124, and two clip members 126 extending distally from the safety cover 122.

In the illustrated embodiment, as shown in FIG. 5, each clip member 126 has an elongated U-shape; however, in other embodiments, the clip members can have any of various shapes including, but not limited to, rectangular, square, triangular, etc. In use, an operator can position a subject between the two clip members 126 to align the subject with the aperture 133 in the lockplate 124 and therefore with the needle 110, such that the needle 110 penetrates the subject 128 at a selected injection location. For fish, the selected injection location can be in the belly between the side fins, as shown in FIG. 5.

In some embodiments, the clip members 126 can be flexible members and can be biased inwardly such that they grip a subject that is positioned between them. For example, the clip members 126 can comprise a self-biasing or elastic material (e.g., steel, plastic, rubber, etc.). In such embodiments, the clip members 126 can be configured to bias into the open or gripping position without the use of a separate biasing member. In other embodiments, the clip members 126 can be pivotably coupled to the safety cover 122 and can be movable between an open position and a gripping position. The clip members 126 in such embodiments can comprise a biasing member (e.g., a spring) configured to bias the clip members into the gripping position. In some embodiments, the clip member 126 can be moved between the open position and the gripping position using, for example, the trigger 103.

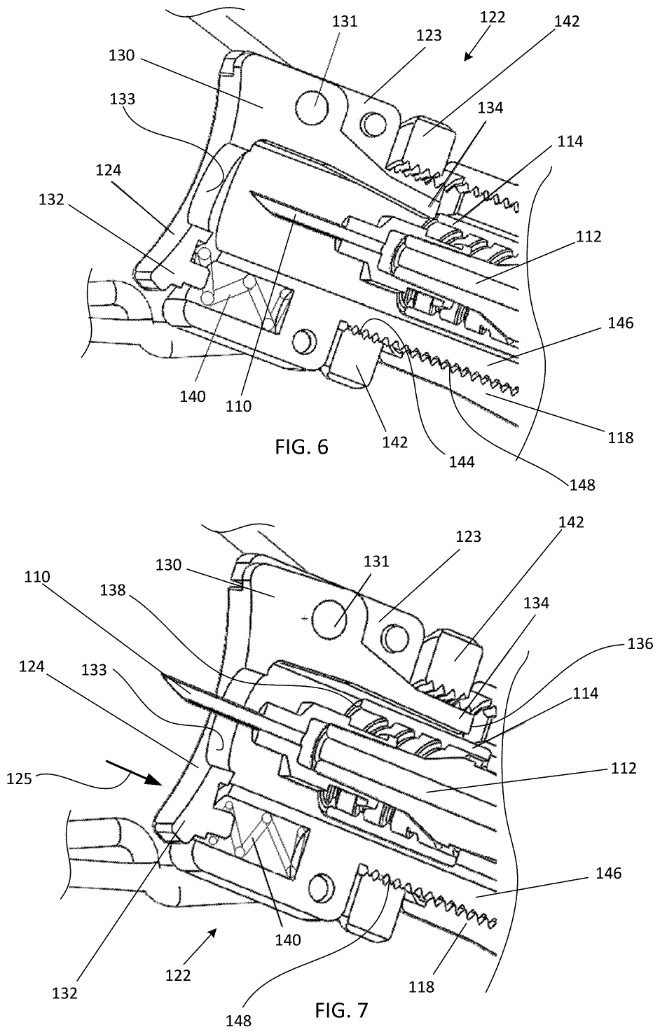

Referring to FIGS. 6-7, the lockplate 124 can be movable between a locked position (FIG. 6) in which safety cover 122 and the movable portion 118 are restrained from movement relative to the needle 110, and an unlocked position (FIG. 7) in which the safety cover 122 and movable portion 118 are movable relative to the needle 110. The lockplate 124 can have a first end portion 130, a second end portion 132, and an aperture 133 extending through a thickness of the lockplate 124. The first end portion 130 can be pivotably coupled to the base 123 via a pivot pin 131 and can comprise a proximally-extending locking member 134. The locking member 134 can have a locking surface 136 (FIG. 7) configured to engage a corresponding locking surface 138 (FIG. 7) to prevent movement of the safety cover 122 and movable portion 118 relative to the needle 110. In the embodiment shown in FIGS. 1-7, the locking surface 138 can be disposed on a distal end portion of the needle support portion 114, as shown in FIG. 4. However, in other embodiments, the locking surface 138 can be disposed on any of various locations configured to prevent movement of the safety cover and movable portion 118 relative to the needle 110.

The second end portion 132 of the lockplate 124 can be coupled to a biasing member 140 (e.g., a spring, a compressible sleeve, etc.). The biasing member 140 is configured to bias the lockplate 124 into the locked position. As shown in FIG. 7, when a proximally-directed force is applied to the second end portion 132, as indicated by arrow 125, the biasing member 140 is compressed and the lockplate 124 pivots about the pivot pin 131, thereby disengaging the locking surfaces 136, 138 from one another and allowing the movable portion 118 and safety cover 122 to move relative to the needle 110 such that the distal point of the needle 110 extends through the aperture 133 and is exposed.

The head portion 102 can further comprise a sensor (e.g., an optical sensor) configured to detect when the needle 110 has reached a selected depth within the subject 128. Once the sensor determines that the selected depth has been reached, it can trigger the injection of a predetermined dose of medicament. In some embodiments, the sensor can further determine whether the full dose of medicament has been injected. For example, if the needle 110 is removed from the subject 128 prior to the full dose being administered, the sensor can trigger an alert letting the operator know that the injection has failed. The alert can be any suitable alert, such as an audible, visual, or tactile alert (e.g., a vibration).

The safety cover 122 can be removably coupled to the movable portion 118 via a connector 142. As shown in FIG. 6, the connector 142 can comprise an internal threaded surface 144. The safety cover 122 can comprise an annular protrusion 146 having a correspondingly threaded external surface 148. To remove the safety cover 122, the safety cover 122 can be rotated in a first direction (e.g., counterclockwise) causing the threads 144 of connector 142 to disengage from the corresponding threads 148 of the annular protrusion 146, thus causing the safety cover 122 to disengage from the movable portion 118. To couple the safety cover 122 (or a replacement safety cover) to the movable portion 118, the safety cover 122 can be rotated in a second direction (e.g., clockwise) causing the threads 144 of connector 142 to engage the corresponding threads 148 of annular protrusion 146, thus coupling the safety cover 122 to the movable portion 118. In other embodiments, the safety cover 122 can be coupled to the movable portion 118 via any suitable connector, such as snap-fit or clip connectors.

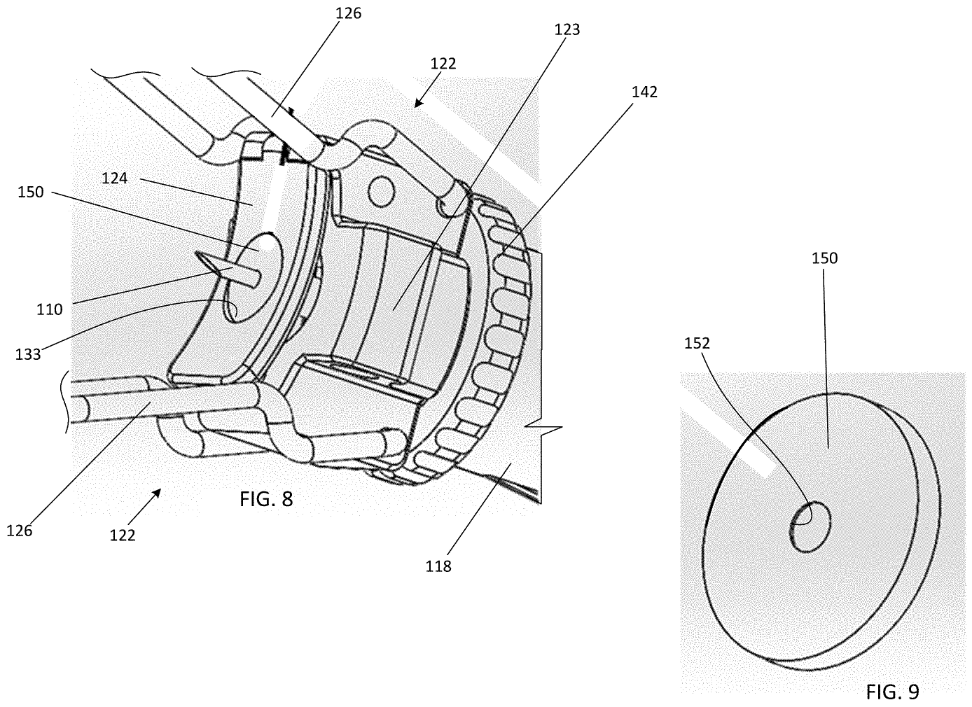

Referring to FIGS. 8-9, in some embodiments, the safety cover 122 can further comprise a removal member 150. The removal member 150 can be coupled to the lockplate 124 and can be configured to remove contaminants such as dirt or scales that adhere to the needle 110. In an illustrated embodiment as shown in FIG. 9, the removal member 150 can be an annular member that includes a needle aperture 152 extending through a thickness of the member 150. The member 150 can be configured (e.g., sized and shaped) to fit within the aperture 133 of the lockplate 124, as shown in FIG. 8. The central aperture 152 can have a diameter slightly wider than an outer diameter of the needle 110, such that as the needle 110 retracts through the needle aperture 152, the inner surface of the needle aperture 152 contacts an outer surface of the needle 110 to remove contaminants (e.g., dirt, scales, etc.) from the surface of the needle 110.

In other embodiments, the aperture 133 of the lockplate 124 can have any of various shapes (e.g., square, rectangular, ovular, triangular, square-oval, etc.) and the removal member 150 can have a corresponding shape such that it can fit within the aperture 133. Though the needle aperture 152 is shown having a circular shape and disposed in the center of the removal member 150 in the embodiment shown in FIGS. 8-9, it should be recognized that the needle aperture can be disposed at any location within the removal member and can have any shape configured to correspond with an outer perimeter of the needle 110.

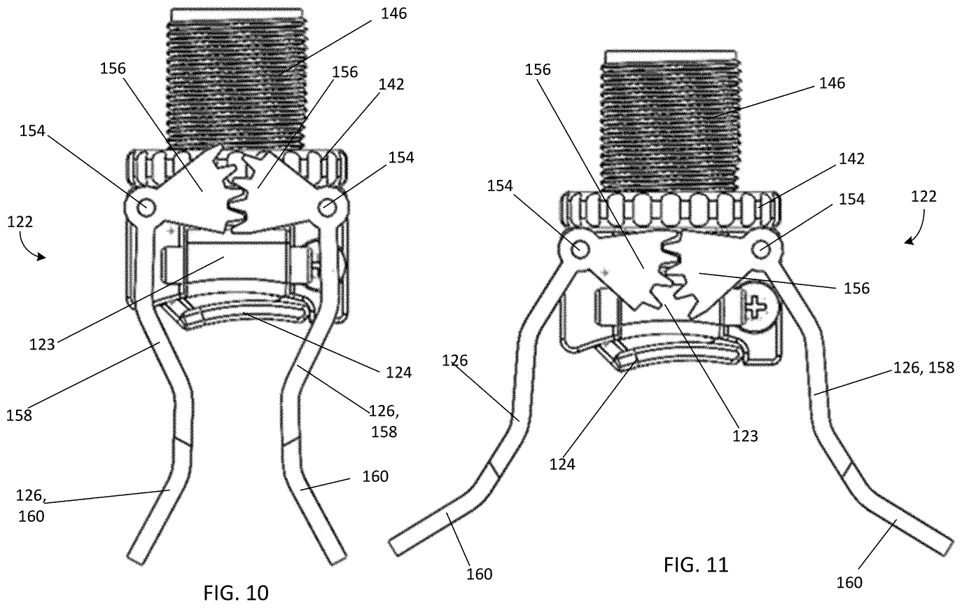

Referring to FIGS. 10-11, in some embodiments, the clip members 126 can be pivotably coupled to the base 123 of the safety cover 122 such that they can move relative to one another about a respective pivot pins 154 between an open position (FIG. 11) and a gripping position (FIG. 10). Each clip member 126 can include a toothed base portion 156 configured to engage a corresponding toothed based portion 156 of an adjacent clip member 126, as shown. The clip members 126 can be coupled to a biasing member (e.g., a spring) configured to bias the clip members 126 into the gripping position or into the open position. Accordingly, once a subject 128 is disposed between the clip members 126, the clip members 126 can retain the subject 128 in the selected position for injection.

As shown, the clip members 126 can have a curved, sinusoidal shape including a first curved portion 158 and a second curved portion 160. In other embodiments, the clip members 126 can have any of various other shapes, for example, the elongated U-shape shown in the embodiment of FIGS. 2-5. In some embodiments, the clip members 126 can comprise a self-biasing or elastic material (e.g., steel, plastic, rubber, etc.). In such embodiments, the clip members 126 can be configured to bias into the open or gripping position without using a separate biasing member.

In still other embodiments, the clip members 126 can be formed such that they can slide relative to one another rather than rotating around a pivot pin 154. For example, each clip member 126 can be slidable toward and away from a longitudinal axis of the hand-held unit 100 (e.g., an axis extending along the length of the needle). The clip members 126 can slide toward one another into a gripping position and/or away from one another into an open position. The clip members 126 can be coupled to a biasing member (e.g., a spring) configured to bias the clip members 126 toward one another into the gripping position. Once a subject 128 is disposed between the clip members 126, the clip members 126 can bias into the gripping position and retain the subject in the selected position for injection.

As mentioned, the head portion 102 can be removably coupled to the hand-held unit 100, which can be coupled to one or more control units 200 and/or one or more medicament containers 300. Each control unit 200 can be configured to deliver a dose of medicament from a respective medicament container 300 to the hand-held unit 100 and into the subject 128. The control unit 200 can comprise at least one pump comprising a dosing chamber fluidly coupled to the one or more containers 300 via a first non-return valve and fluidly coupled to the hand-held unit by a second non-return valve. A dose of medicament can enter the dosing chamber from the medicament container 300 via the first non-return valve, and exit the dosing chamber to the hand-held unit 100 via the second non-return valve. The pump can be configured to push/pull the same or different amount (e.g., dosage) of medicament from each container 300 and deliver those amounts to the hand-held unit 100. In some particular embodiments, the injection apparatus 10 can be designed to deliver a dose of about 0.05 ml to about 3 ml of medicament to each subject, in each injection.

In some embodiments, each control unit can comprise two or more pumps, wherein the number of pumps is identical to the number of containers 300, and wherein each pump is coupled to a discrete container 300. In such configurations, each pump is designed to pull and/or push a predetermined amount of medicament from its respective container 300 and to sequentially deliver the medicament to the hand-held unit 100 and into the subject according to a predefined administration order.

Referring again to FIG. 1, the control unit 200 can further comprise a control panel 204. The control panel 204 can comprise an input device (e.g., a keypad and/or touchscreen) and/or a display configured to display information about the injection process. In some embodiments, the control panel 204 can be actuated manually by a user. Alternatively, the control panel can be controlled remotely via a remote device (e.g., a handheld mobile device using a smartphone or tablet app). The control unit 200 can be configured to receive and store a selected medicament volume inputted by an operator using the control panel and can adjust the motor and pump such that the selected medicament volume is injected for each injection.

The injection apparatus 10 can further comprise a power source configured to allow the injection apparatus 10 to be suitable for remote locations with limited power supplies. Any suitable power source can be used including, for example and without limitation, a battery pack, a solar panel, a hydrogen fuel cell, etc. Combinations of power sources can also be used, where the power sources can be the same, such as two battery packs, or different, such as a solar panel and a battery pack. In some embodiments, the power source can be rechargeable. In other embodiments, the power source can be disposable (e.g., disposable batteries). The power source can be removably coupled to the control unit 200 and can be configured to provide power to the injection apparatus 10. In other embodiments, the power source can be coupled to the hand-held unit 100, to a container 300, or can be a separate piece that is operatively coupled to the injection apparatus via one or more power cables. In still other embodiments, the hand-held unit 100 can comprise a second power source configured to provide power to the hand-held unit.

The control unit 200 can further comprise one or more cables integral therewith or couplable thereto. In the illustrated embodiment, cable 206 operatively couples the control unit 200 to the hand-held unit 100. The cable 206 is configured to provide power to the hand-held unit 100 and/or to allow for communication between the hand-held unit 100 and the control unit 200. For example, the hand-held unit 100 can send a signal to the control unit 200 when the needle 110 is in position to make an injection.

In some embodiments, the injection apparatus 10 can be configured to inject two or more different medicaments into a subject at the same location, or in two or more different locations using a single needle. In such embodiments, the injection apparatus can, for example, comprise three main subsystems: (1) a hand-held unit 100 comprising a single needle; (2) one or more control units 200 each comprising a pump, one or more non-return valves, and a power source; and (3) two or more containers 300 containing different medicaments. Further details of this configuration can be found, for example, in WO 2018/203203, which is incorporated by reference herein in its entirety. In other embodiments, the injection apparatus 10 can comprise a plurality of needles each fluidly coupled to a respective pump coupled to a respective medicament container.

Though the injection apparatus 10 is described with reference to intramuscular injections (e.g., injections that are perpendicular to the surface of the subject), it should be understood that the head portion 102 and safety cover 122 can be adapted for subcutaneous injection as well (e.g., by modifying the angle of the lockplate 124 and/or removing or modifying the clip members 126). Subcutaneous injections require that the needle penetrate the subject's skin but stop prior to penetration of the subject's muscle such that the medicament is deposited between the skin and the muscle. Such injections require that the needle enter almost parallel to the skin. As used herein, unless stated in absolute terms such as “completely parallel,” or “completely perpendicular” the terms “parallel” and “perpendicular” include the terms “substantially parallel” and “substantially perpendicular.” For example, an object is substantially parallel with respect to a reference object or plane when the object is oriented at an angle of ±20° or less with respect to the reference object or plane, and is substantially perpendicular with respect to a reference object or plane when the object is oriented at an angle of ±20° or less with respect to the reference object or plane.

In some particular embodiments wherein the injection apparatus 10 is being used for subcutaneous injection, the head portion 102 can be removed to expose the needle. In such embodiments, manual injection can be used in lieu of automatic injection. For example, the operator can insert the needle into the subject substantially parallel to the subject's skin (or scale) surface and can actuate the trigger 103 to activate the pump and inject the medicament.

Generally, the medicaments are administered in liquid form. In some embodiments, the containers 300 are provided with a ready-for-use liquid medicament. In other embodiments, the medicament to be administered is administered in a dry form (e.g., as a sprayed powder). In such embodiments the medicament is held within the containers in a dry form. In still other embodiments, the containers 300 are provided with a medicament (e.g., in a dried or powdered form) that needs to be processed or prepared prior to use (e.g. by the addition of water, alcohol, or other solvent or suspension aid thereto). Accordingly, in some embodiments, the containers 300 can be internally divided into two or more compartments for holding one or more powdered medicaments and one or more solvents or suspension aids.

As shown in FIG. 1, the containers 300 can be formed separately and can be removably couplable to the at least one control unit 200 via one or more connecting tubes 202. In some embodiments, each control unit 200 can be fluidly coupled to a respective container 300. The containers can either be refilled or replaced with full containers when emptied. In some embodiments, the containers 300 can be formed integrally with or couplable to the hand-held unit 100, the control unit 200, or both, and can be refilled with a suitable liquid medicament when emptied.

As mentioned previously, the control unit 200 can be configured to receive data from and/or transmit data to a remote device. The remote device can be configured to store data from, send data to, and/or remotely control the injection apparatus 10. The remote device can be, for example, a general-purpose computer, a hand-held mobile device (e.g., a cell phone or tablet), and/or any type of accessory therefore (e.g., a “smart watch” etc.). The remote device can include an application, or “app,” configured to control the administration process and/or track information relating to the administration process. In some embodiments, the control unit 200 can transmit real-time information to the remote device which information can be displayed by the app. In some embodiments, multiple control units 200 from multiple injection apparatuses 10 can transmit real-time information to the same remote device.

Further details of the power source, control unit 200, medicament chambers 300, remote device, computing environment, and graphical user interface can be found, for example, in International Application No. PCT/IB2020/053177.

The injection apparatus 10 can be configured to operate in and switch between various injection modes. For example, the injection apparatus 10 can operate in an automatic mode, a manual mode, and/or a semi-automatic mode. An operator can switch between modes by, for example, changing the firmware mode. In some embodiments, the mode can be selected by entering a command on the control unit 200 (e.g., by pressing a ‘mode selection’ button), in other embodiments, the mode can be selected using a remote device including an application or “app” configured to control the administration process.

Operating in automatic mode, an injection apparatus 10 comprising head 102 can be used to administer medicament to a subject in the following exemplary manner. The operator can actuate the trigger 103 to release the lock, allowing the movable portion 118 to move axially relative to the support portion 116. The operator can position a subject 128 between the clip members 126, as shown in FIG. 5, and urge the subject 128 against the lockplate 124 at the injection site (e.g., the subject's belly and/or between the subject's fins). As the subject 128 is urged against the lockplate 124, the lockplate 124 pivots around the pivot pin 131 such that the locking surfaces 136, 138 (FIG. 7) disengage from one another and the safety cover 122 and movable portion 118 move relative to the needle 110, thereby exposing the needle 110 and allowing the distal point of the needle 110 to pierce the subject 128. One the distal point of the needle 110 reaches a selected depth within the subject 128 (e.g., as determined by the sensor), the injection apparatus 10 can automatically inject the medicament. Once the dose of medicament has been injected (which can be indicated by some sort of indicia, e.g., a green light or a sound), the operator can withdraw the needle 110 from the subject 128.

When switching the injection apparatus to manual mode, the operator can select the ‘manual’ firmware mode and can secure the movable portion 118 and the safety cover 122 in the retracted position (e.g., such that the distal point of the needle 110 is exposed from the movable portion 118 and the safety cover 122). When operating in the ‘manual’ mode, the injection apparatus 10 will inject medicament whenever the trigger 103 (FIG. 1) is actuated, regardless of the position of the lockplate 124 and/or of the subject 128.

The injection apparatus 10 can also be operated in a ‘semi-automatic’ mode, wherein the injection apparatus 10 will inject medicament only if both the trigger 103 and the lockplate 124 are actuated simultaneously. When operating in semi-automatic mode, the operator can actuate the trigger 103 to inject the medicament. For example, the subject 128 can be disposed between the clip members 126 and urged against the lockplate such that the needle 110 enters the subject at a selected injection site. After the needle is inserted into the subject, the operator can actuate the trigger 103 to inject the medicament into the subject. Such an operation mode advantageously allows the operator to control the timing of the injection and to inject only if the operator determines that the needle is in the selected position.

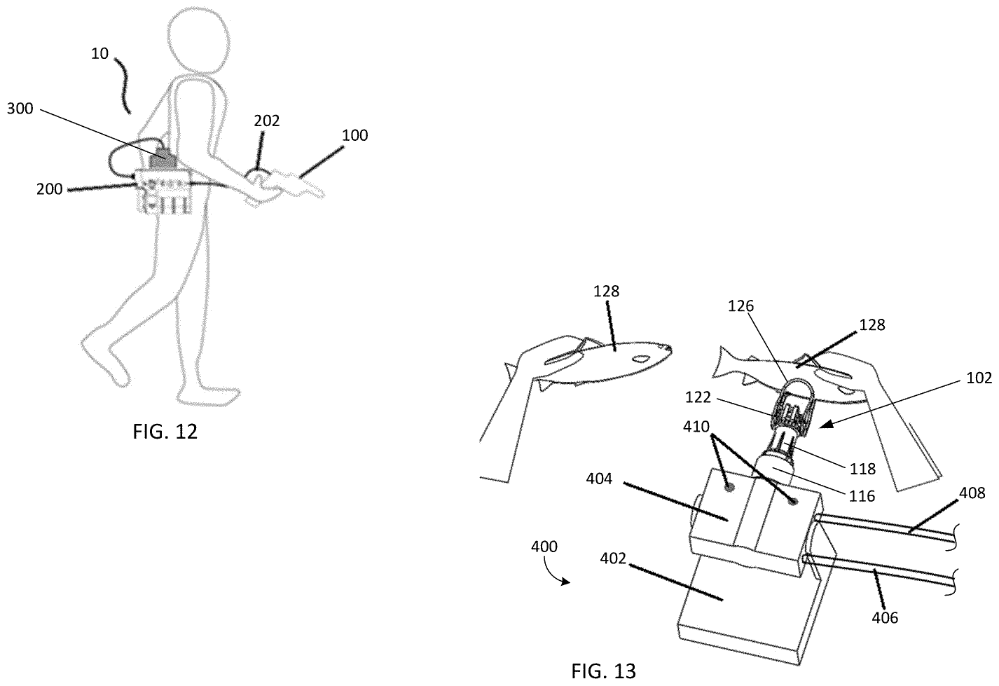

As shown in FIG. 12, in some embodiments, the injection apparatus 10 can be worn and/or carried by an operator. In such embodiments, the one or more control units 200 can be configured to be worn by the operator, such as on a belt, in a waist bag, a backpack, a vest, or in a carryon bag. In other embodiments, the one or more control units 200 can be directly coupled to or formed integrally with the hand-held unit 100. The medicament container(s) 300 can also be worn and/or carried by the operator.

The connecting tubes 202 can be flexible enough to allow the operator to move the hand-held unit 100, long enough to allow the operator to fully extend his/her arm holding the hand-held unit 100, and rigid enough (i.e., non-expandable and non-deformable) to prevent widening of the tube due to pressure caused by medicament passing through the tube. Pressure changes have the potential to deform a tube that lacks sufficient rigidity during an administration of medicament, which can lead to inaccurate dosing of the medicament or a delay between the pump action and the administrating of the medicament to the animal. In some embodiments, the connecting tube 202 can flex in all directions and can withstand twisting. In some embodiments, the connecting tube can elastically return to its original shape after being bent, twisted, extended, or otherwise deformed. Further details of the connecting tubes and wearable configuration can be found, at least, in International Application No. PCT/IB2020/053177.

The injection apparatus 10 can be configured to be submersible in water (e.g., waterproof and/or water resistant) such that at least the hand-held unit 100 can be submerged in an aquatic environment, such as a tank or aquatic pen. In such embodiments, the operator can submerge the hand-held unit 100 in water such that an aquatic subject is disposed between the clip members 126, and can urge the lockplate 124 against the subject to trigger an injection. Such a configuration allows the operator to easily inject a large number of aquatic subjects without having to remove the subjects from the water, which can cause panic, flailing, or potential injury.

Referring to FIG. 13, in other embodiments, the injection apparatus can be configured as a base station injection apparatus 400 to which the head portion 102 can be removably coupled. The base station 400 can comprise a base plate 402 couplable to a table or other surface, and an upper member 404 coupled to the base plate 402 and comprising a needle extending from the upper member 404. The head portion 102 comprising support portion 116, movable portion 118, and safety cover 122 can be removably coupled to the upper member 404.

The upper member 404 can be fluidly coupled to the control unit 200 via a flexible connecting tube 406, similar to tubes 202 described previously, and can be operatively coupled to the control unit 200 via a communication cable 408. The upper member 404 can receive medicament from the medicament containers 300 via the control unit 200 and the connecting tube 406. The upper member 404 can communicate (e.g., receive and/or transmit data, commands, etc.) with the control unit 200 via the communicating cable 408. In other embodiments, the upper member 404 can communicate wirelessly with the control unit 200 and/or a remote unit. As mentioned previously, the control unit 200 can comprise at least one pump comprising a dosing chamber fluidly coupled to the one or more medicament containers 300

The base station apparatus 400 can be configured to operate in the following exemplary manner. The operator can grab a first subject 128, position the subject 128 between the clip members 126, and urge the subject 128 against the lockplate 124 of the safety cover 122, as shown in FIG. 13. The subject 128 can be urged against the lockplate 124 such that the injection site (e.g., the subject's belly) is aligned with the needle. As the subject 128 is urged against the lockplate 124, the lockplate 124 pivots around the pivot pin 131 such that the locking surfaces 136, 138 (FIG. 7) disengage from one another and the safety cover 122 and movable portion 118 can move axially relative to the needle, thereby exposing the needle and allowing the distal point of the needle to pierce the subject 128. One the distal point of the needle 110 reaches a selected depth within the subject 128 (e.g., as determined by the sensor), the injection apparatus 400 can automatically inject the medicament. Once the dose of medicament has been injected, the operator can withdraw the needle from the subject 128 and can release the subject. The operator can then grab a second subject 128 and repeat the process.

In another embodiment, the base station 400 can be configured to be utilized without the lockplate 124 and/or movable portion 118. In such embodiments, the operator can urge the subject against the needle and the sensor (e.g., a mechanical sensor, an optical sensor, an electrical sensor, and/or an inductance sensor) of the base station 400 can detect when the needle has reached a selected depth within the subject 128 and the injection apparatus can automatically inject the medicament.

The base station 400 can further comprise one or more indicators 410 (e.g., LED lights) configured to communicate information about the injection process. For example, the indicators can indicate whether the injection was successful, whether the apparatus 400 is ready for a subsequent injection, whether an error and/or malfunction has occurred, etc. In the embodiment shown in FIG. 13, the indicators 410 are visual indicators such as LED lights. However, in other embodiments, in lieu of or in addition to visual indicators, the indicators can comprise any suitable indicator, such as tactile indicators (e.g., vibrations) and/or audible indicators.

The base station apparatus 400 can be configured to be submersible in water such that it can be disposed in an aquatic environment, such as a tank or aquatic pen. In such embodiments, the operator can stand in the water and guide the subject 128 through the water such that the subject is positioned between the clip members 126, briefly urge the subject against the lockplate 124 to trigger an injection, and then release the subject 128. Such a configuration allows the operator to easily inject a large number of aquatic subjects without having to remove the subjects from the water, which can cause panic, flailing, or potential injury.

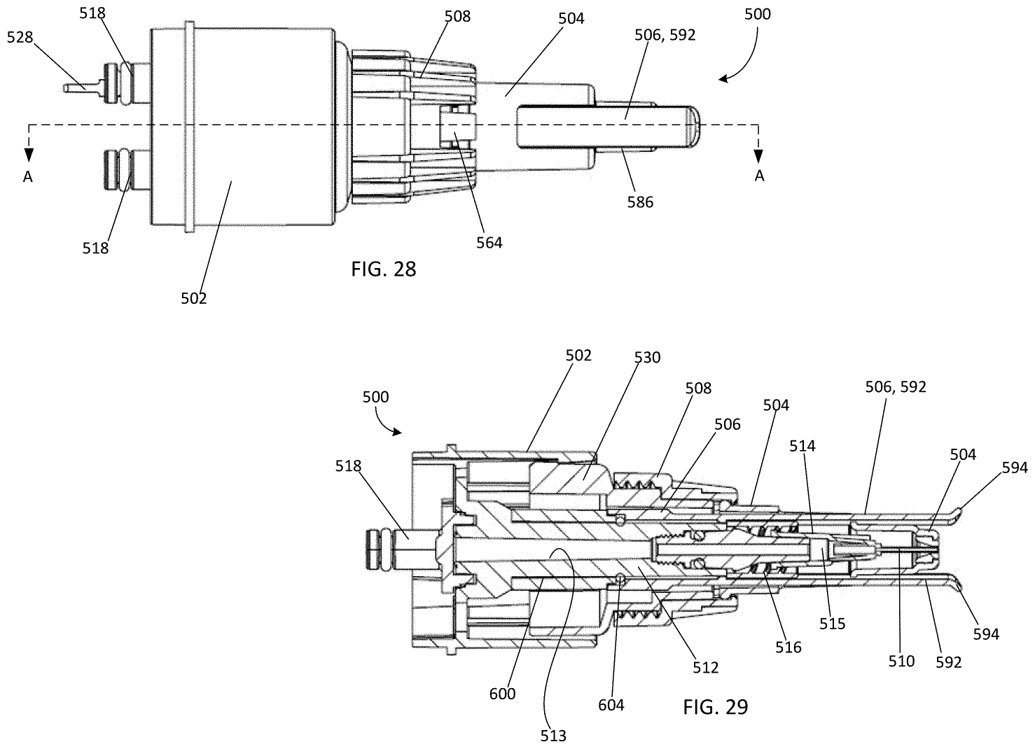

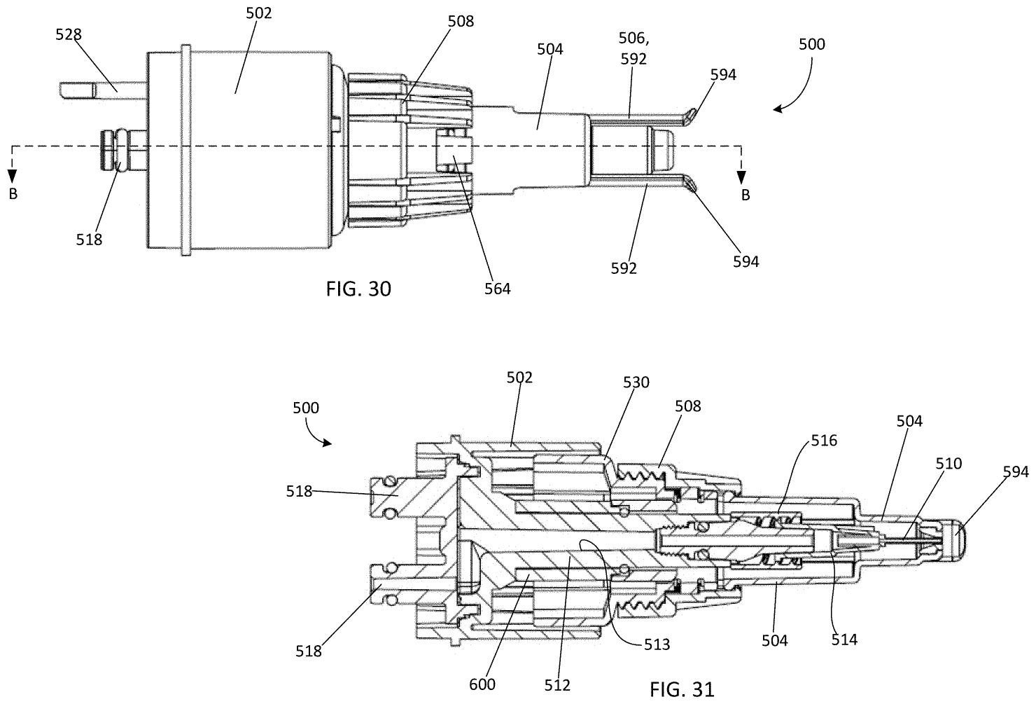

FIGS. 14-31 illustrate another embodiment of a head portion 500 that can be coupled to a body portion (e.g., such as body portion 104 described previously) to form the hand-held unit 100 of a modular injection apparatus 10 for injecting one or more medicaments into a subject. As described previously with respect to the embodiments of FIGS. 1-12, the injection apparatus 10 can further comprise at least one control unit 200 fluidly coupled to the hand-held unit 100 and one or more medicament containers 300 removably coupled to the at least one control unit 200.

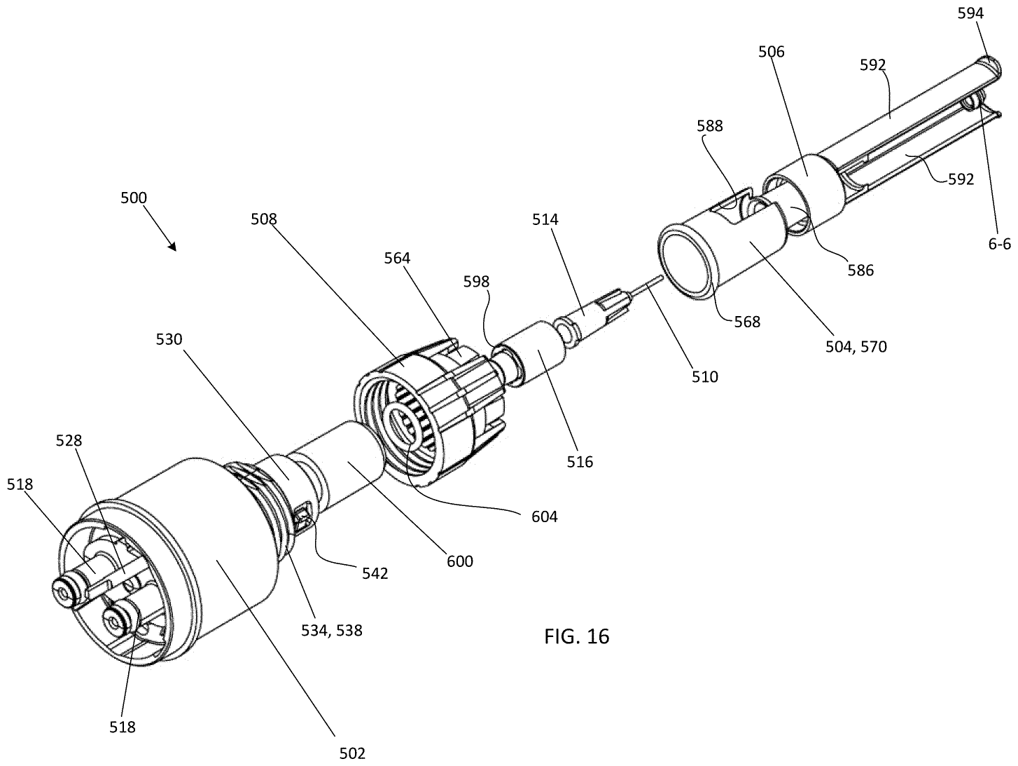

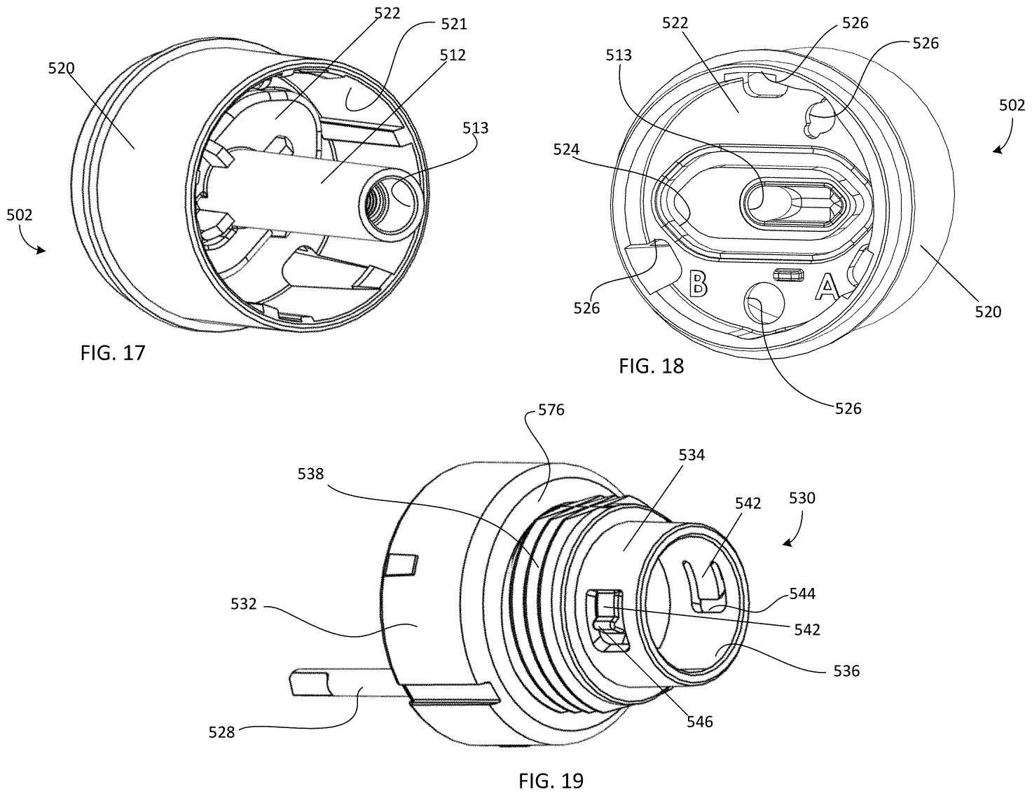

The head portion 500 can generally comprise a support portion/base member 502, a movable portion 504, a safety cover 506, an adjustment member 508, and one or more needles 510 (FIG. 16) configured to administer medicament to the subject. In the illustrated embodiment shown in FIGS. 14-31, the head portion 500 comprises one needle 510; however, in other embodiments, the head portion 500 can comprise two or more needles, each of which can be fluidly coupled to the same pump or to respective pumps. Referring to FIG. 16, which shows an exploded view of head 500, the needle 510 can be fluidly coupled to a connecting tube 512 (FIG. 17) via a needle hub 514 comprising a luer lock 515 (FIG. 29). A needle support 516 can surround and support the needle hub 514. An inner bore 513 of the connecting tube 512 can be fluidly coupled to one or more inlets 518, which can be fluidly coupled to the medicament containers 300 via the body portion 104 of the hand-held unit 100 and the control unit 200.

Referring to FIGS. 17-18, the base member 502 can comprise a body 520 having an inner bore 521 (e.g., an annular bore) and an endplate 522 from which the extension member/connecting tube 512 extends. As shown in FIG. 17, the connecting tube 512 can comprise an internal threaded portion configured to couple the luer lock 515 such that the needle 510 is fluidly coupled to the inner bore 513. In the embodiment shown in FIGS. 14-31, the body is cylindrical, however, in other embodiments, the body 520 can have any of various shapes such as cuboid, ovular, etc. As shown in FIG. 18, the end plate 522 can comprise a recess 524 in which the inlets 518 can be disposed. In some embodiments, such as the illustrated embodiment, the end plate 522 can comprise various apertures 526 through which components of the head portion 500 or body portion 104 can extend, for example, to couple the head portion 500 to the body portion 104, or to allow a portion of a the head portion (such as second extension member 528) to extend into the body portion 104.

The head portion 500 can further comprise a support member 530 configured to be disposed within the inner bore 521 of the base member 502. The support member 530 can be movable relative to the base member 502 such that the support member 530 moves when the movable portion 504 is actuated. A biasing member (e.g., a spring or polymeric sleeve) can be disposed within the inner bore 521 of the base member 502 between the base plate 522 and the support member 530 in order to bias the support member 530 (and therefore the movable portion 504) into the extended position.

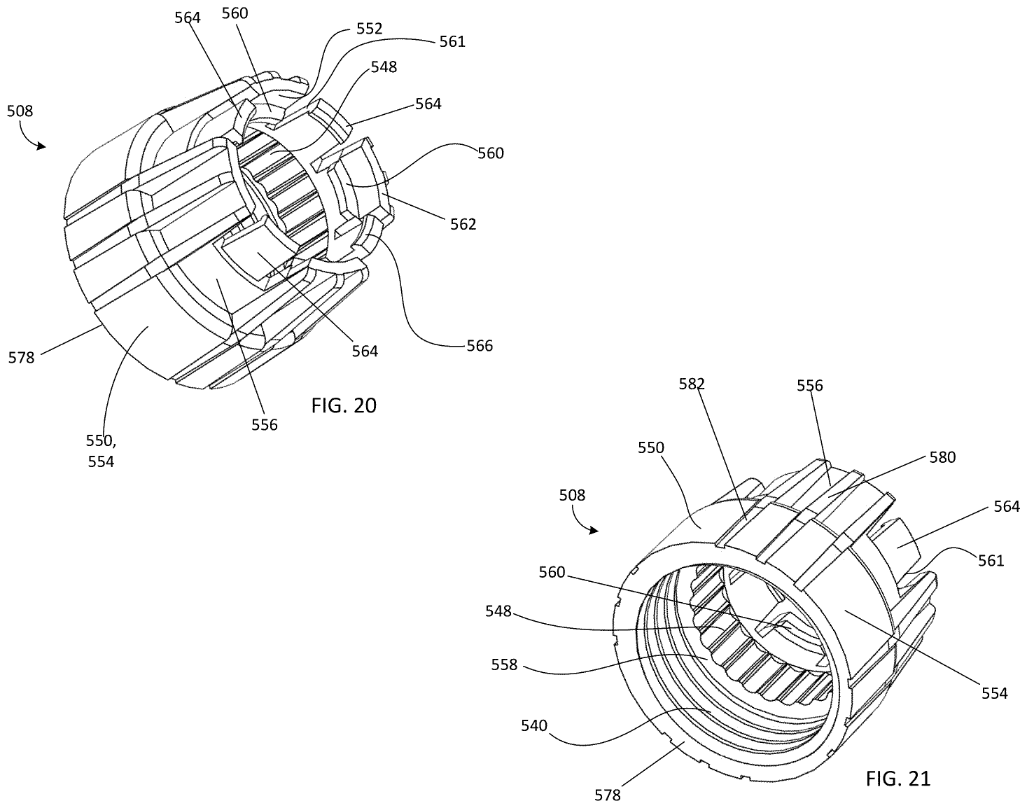

The support member 530 can comprise a base portion 532, an extension member 534 extending from the base portion 532, and a central lumen or bore 536 extending through the support member 530. The extension member 534 can comprise an external threaded portion 538 configured to mate with an internal threaded portion 540 (FIG. 21) of the adjustment member 508. The extension member 534 can further comprise one or more resilient clips or latches 542 disposed within a cutout 544. Each latch 542 can be configured to engage a ridged portion 548 (FIG. 20) of the adjustment member 508. The latches 542 can be biased radially outwardly such that a protrusion 546 extending from the latch is urged into engagement with the ridged portion 548. The engagement of the latches 542 with the ridged portion 548 can advantageously prevent inadvertent rotation of the adjustment member 508 relative to the support member 530, which prevents inadvertent adjustment of the needle depth. For example, the latches 542 can be configured to restrain the adjustment member 508 against rotation relative to the support member 530 unless a rotational force above a selected threshold (e.g., greater than the biasing force of the latches 542) is applied to the adjustment member.

The support member 530 can further comprise a second extension member or rod 528 coupled to a side wall of the base portion 532 and extending in a direction opposite the first extension member 534. The second extension member 528 can be configured to engage a sensor (e.g., a photointerruptor) disposed in the body portion 104 of the hand-held device 100 in order to automatically trigger injection of medicament. For example, when the movable portion 504 is actuated, it moves the support member 530 such that second extension member 528 engages the sensor. Once engaged, the sensor can trigger the injection of a predetermined dose of medicament.

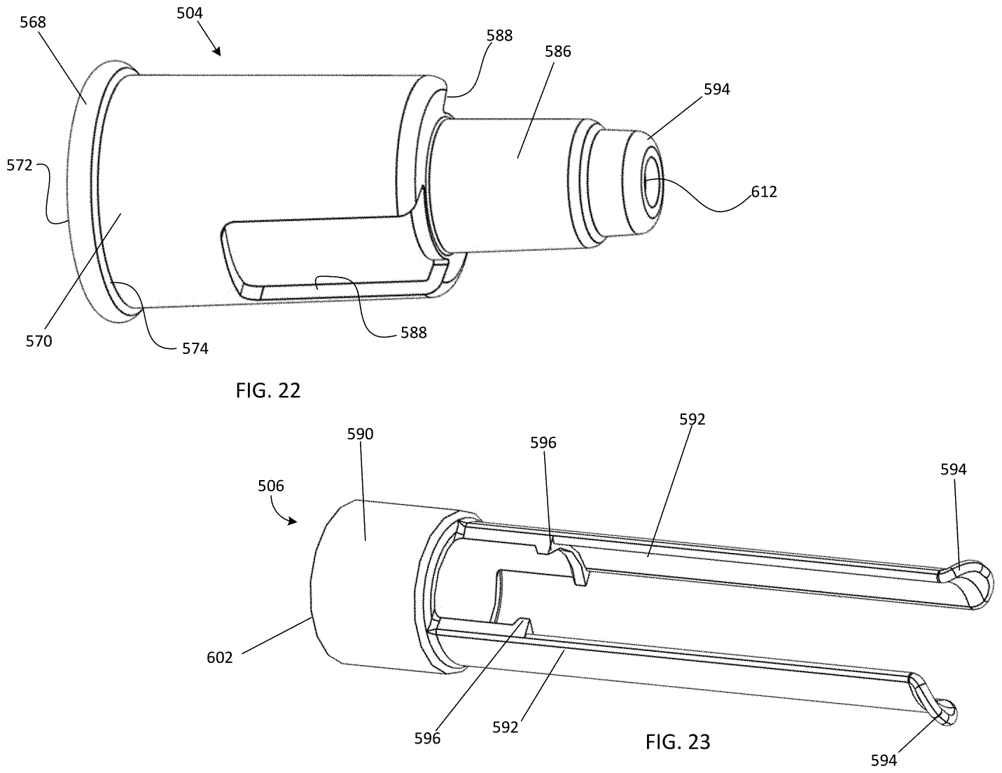

Referring to FIGS. 20-21, as mentioned, the head portion 500 can comprise an adjustment member 508 coupled to the support member 530. The adjustment member 508 can comprise a body 550 configured as a substantially cylindrical member defining an inner bore 552. The body 550 can have a first end portion 554 having a first inner diameter and a second end portion 556 having a second inner diameter smaller than the first inner diameter. The first and second portions 554, 556 can be separated by a shoulder 558 within the inner bore 552. The second end portion 556 can comprise one or more protrusions 560 extending radially into the inner bore 552 and axially spaced from a distal edge 562 of the adjustment member 508. The second end portion can further comprise one or more clip members 564 each having a radially-extending lip portion 566 axially spaced from the protrusions 560. The clip members 564 can be separated from the protrusions 560 by one or more channels 561 extending axially from the distal edge 562 of the adjustment member 508. The protrusions 560 and clip members 564 can be configured to engage an annular lip/shoulder 568 (FIG. 22) disposed on a first or proximal end portion 570 of the movable portion 504 in order to releasably couple the movable portion 504 to the adjuster 508. Referring to FIG. 22, a first or proximal edge 572 of the shoulder 568 can engage the protrusions 560, and a second or distal edge 574 of the shoulder can engage the clip members 564. The clip members 564 can be resilient such that they can bend or deform radially outwardly when the movable portion 504 is moved distally with sufficient force. Such a configuration advantageously allows the user to remove the movable portion 504 (e.g., to replace the movable portion 504 or the needle 510) without removing or otherwise adjusting the location of the adjustment member 508, thereby prevents or mitigates inadvertent adjustment of the needle penetration depth. For example, the user can pull on the movable portion 504 to remove it from the adjustment member 508 without affecting the position of the adjustment member 508.

The adjustment member 508 can be used to adjust the penetration depth of the needle 510. A user can rotate the adjustment member 508 to increase or decrease the distance between the support member 530 and the movable portion 504. For example, by increasing the distance between an outer shoulder 576 (FIG. 19) of the support member 530 and a proximal edge 578 of the adjustment member 508, thereby adjusting the distance that the movable portion 504 can retract. The greater the distance between the outer shoulder 576 and the proximal edge 578, the shallower the needle penetration depth. The smaller the distance between the outer shoulder 576 and the proximal edge 578, the deeper the needle penetration depth. As mentioned previously, the latches 542 of the support member 530 can engage the inner ridged portion 548 of the adjustment member 508 such that a user must overcome the biasing force of the latches 542 in order to rotate the adjustment member relative to the support member 530, thereby preventing inadvertent rotation of the adjustment member.

The outer surface of the adjustment member 508 can comprise a gripping portion configured to allow a user to more easily grip and manipulate the adjustment member 508. The gripping portion can comprise, for example, a plurality of ridges 580 and channels 582. In other embodiments, the gripping portion can comprise bumps, knurling, or other texture to facilitate gripping by the user.

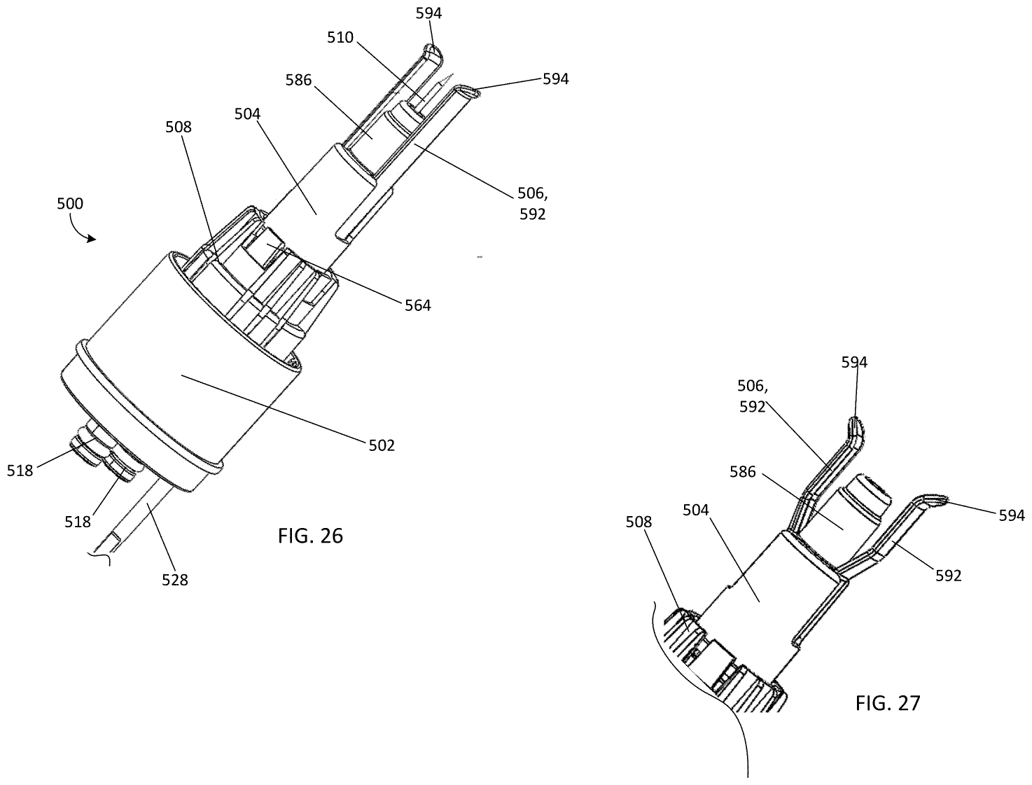

Referring to FIG. 14, as mentioned, the head portion 500 can comprise a movable portion 504, which can be movable relative to the base member 502 in a telescoping manner (e.g., as represented by arrow 584). The movable portion 504 can be movable between an extended position (see e.g., FIG. 14) wherein the distal point of the needle 510 is covered by the movable portion 504, and a retracted position (see e.g., FIG. 26) wherein the distal point of the needle 510 is exposed. As mentioned previously, in some embodiments, the head portion 500 can further comprise a biasing member (e.g., a spring, compressible sleeve, etc.) configured to bias the movable portion into the extended position for safety. The needle support 516/needle hub 514, and therefore the needle 510, can be coupled to the base member 502 (or in some embodiments to the body portion 104) such that the movable portion 504 can move relative to the needle 510.

Urging the movable portion 504 rearwardly (e.g., proximally) relative to the base member 502 exposes a distal point of the needle 510 and allows the needle to be inserted into the subject's body. In some embodiments, urging the movable portion 504 rearwardly into the retracted position (e.g., by pressing the movable portion 504 against a selected injection location on the subject) triggers the release of a predetermined dose of medicament (e.g., by opening a valve associated with the needle and/or by triggering movement of a pump within the control unit 200). Further details of movable portions and base members can be found, for example, in International Application No. PCT/IB2020/053177.

As shown in FIG. 22, the movable portion 504 can have a first or proximal end portion 570 and a second or distal end portion 586. The first end portion 570 can have a diameter greater than a diameter of the first end portion, such that the movable portion 504 has a stepped configuration. The first end portion 570 can comprise first and second opposing apertures 588 extending through the wall of the first end portion 570. The apertures 588 can have an elongated substantially rectangular shape configured to allow the extension members 592 of the safety cover 506 to extend through the apertures 588 such that the movable portion 504 can move relative to the safety cover 506 to expose the distal end of the needle 510.

Referring to FIG. 23, the safety cover 506 can comprise a base portion 590 (e.g., an annular base portion) and one or more extension members 592 extending from the base portion 590. The extension members 592 can be configured to extend past a distal end 594 of the movable portion 504 when the movable portion is in the extended position, as shown in FIG. 14. In the illustrated embodiments, the safety cover 506 comprises two extension members, however in other embodiments the cover 506 can comprise any number of extension members. The safety cover 506 can be coupled to the stationary portion 502 such that force applied to the safety cover 506 does not cause the movable portion 504 to retract. The safety cover 506 can be configured to prevent or mitigate accidental injection by preventing the movable portion 504 from being actuated unless the subject is in the selected injection position (e.g., between the extension members 592).

In the embodiment shown in FIGS. 14-26, the extension members 592 are elongated rectangular members having a curved C-shape in cross-section. However, in other embodiments, the extension members 592 can have any of various other shapes such as elongated members having a sinusoidal curve, triangular members, square members, etc. For example, FIG. 27 illustrates an embodiment of the safety cover 506 for use with larger subjects. The extension members 592 in such an embodiment can have a sinusoidal S-shape. The extension members 592 can each comprise a flared tip portion 594 extending radially away from a longitudinal axis of the head portion 500 (e.g., an axis extending along the length of the needle 510). The flared tip portions 594 can facilitate insertion of a subject between the extension members 592.

Each extension member 592 can comprise a lip portion 596 that extends toward a central longitudinal axis of the head 500. A proximal edge 598 (FIG. 16) of the needle hub 516 can engage the lip portions 596 to further restrain the needle 510 against movement relative to the base member 502. The head portion 500 can further comprise an additional base member or gripper 600 (FIG. 16) against which a proximal edge 602 of the safety cover 506 can abut. The gripper 600 can be disposed within the inner bore 521 of the base member 502 around the connecting tube 512. In some embodiments, such as shown in FIG. 16, an O-ring 604 (FIG. 16) can be disposed between the gripper 600 and the safety cover 506 to help secure the components to one another.

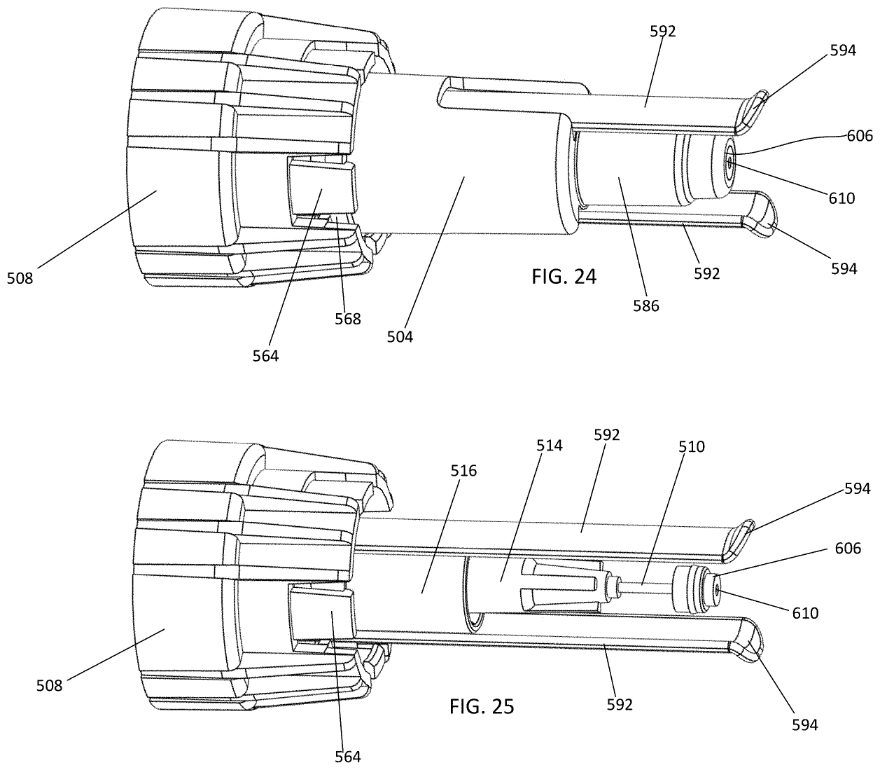

Referring to FIG. 25, in some embodiments, the head portion 500 can further comprise a removal member 606. The removal member 606 can be configured to remove contaminants such as dirt or scales that adhere to the needle 510. In an illustrated embodiment as shown in FIG. 25, the removal member 606 can be an annular member that includes a needle aperture 610 extending through a thickness of the member 606. The removal member 606 can be configured (e.g., sized and shaped) to fit within a distal aperture 612 (FIG. 22) of the movable portion 504, as shown in FIG. 24. The needle aperture 610 can have a diameter slightly wider than an outer diameter of the needle 510, such that as the needle 510 retracts through the needle aperture 152, the inner surface of the needle aperture 152 contacts an outer surface of the needle 110 to remove contaminants or debris (e.g., dirt, scales, etc.) from the surface of the needle 110.

In some particular embodiments, the needle 510 can be an 18 gauge needle. In other embodiments, the needle 510 can be between 20 gauge and 23 gauge.

An injection apparatus comprising head portion 500 can be used in the following exemplary manner. An operator or user can position a subject (e.g., a fish) between the extension members 592 to align the subject with the distal end 594 of the movable portion 504 (and therefore with the distal point of the needle 510). The user can urge the subject against the movable portion 504 such that movable portion 504 moves to the retracted position (see FIG. 26), and such that the needle 510 penetrates the subject at the selected injection location. Once the needle 510 has reached a selected penetration depth (as determined by the interaction between the second extension member 528 and the sensor), the control unit can automatically actuate the pump and thereby inject a dose of medicament into the subject. In other embodiments, the medicament can be injected manually (e.g., by pressing a trigger or other actuation mechanism to actuate the pump). The user can then release the subject/remove the subject from between the extension members, allowing the movable portion 504 to bias into the extended position and the removal member 606 to remove any contaminants or debris from the surface of the needle. For subjects that are fish, the selected injection location can be, for example, in the belly between the side fins. In some particular embodiments, the subjects can be fish up to 125 grams in size. In still other embodiments, the subjects can be fish 2000 grams or larger in size.

In view of the many possible embodiments to which the principles of the disclosure may be applied, it should be recognized that the illustrated embodiments are only preferred examples and should not be taken as limiting the scope. Rather, the scope is defined by the following claims. We therefore claim all that comes within the scope and spirit of these claims.

We claim:

1. An injection apparatus, comprising:
a hand-held unit having a head portion and a body portion, the head portion comprising
a base member,
a needle fluidly coupled to the base member,
a movable portion disposed over the needle and being axially movable relative to the base member between an extended position and a retracted position in which a distal point of the needle is exposed,
a safety cover removably coupled to the base member, the safety cover comprising one or more extension members, and
an adjustment member removably coupled to the movable portion and configured to adjust a penetration depth of the needle by increasing or decreasing a distance between the movable portion and the base member.

2. The injection apparatus of claim 1, further comprising a support member disposed within an inner bore of the base member, the support member comprising a threaded extension member rotatably coupled to a correspondingly threaded interior surface of the adjustment member.

3. The injection apparatus of claim 2, wherein the support member further comprises one or more resilient latches engaging a ridged portion disposed on an inner surface of the adjustment member.

4. The injection apparatus of claim 3, wherein the engagement of the latches with the ridged portion restrains the adjustment member against rotation relative to the support member unless a rotational force above a selected threshold is applied to the adjustment member.

5. The injection apparatus of claim 1, wherein the one or more extension members extend past a distal end of the movable portion when the movable portion is in the extended position.

6. The injection apparatus of claim 1, wherein medicament is administered automatically upon insertion of the needle to a selected depth within a subject.

7. The injection apparatus of claim 1, wherein the movable portion comprises one or more apertures through which the one or more extension members of the safety cover extend.

8. The injection apparatus of claim 1, further comprising a removal member coupled to the movable portion, the removal member comprising an aperture through which the distal point of the needle extends when the movable portion is in the retracted position.

9. The injection apparatus of claim 1, further comprising a biasing member disposed between the base member and the support member to bias the movable portion into the extended position.

10. The injection apparatus of claim 1, further comprising at least one control unit fluidly coupled to the hand-held unit and one or more medicament containers fluidly coupled to the control unit.

11. The injection apparatus of claim 1, wherein the adjustment member comprises one or more clip members and one or more protrusions configured to releasably couple the adjustment member to the movable portion.

12. The injection apparatus of claim 11, wherein a proximal end portion of the movable portion comprises an annular shoulder disposed between the clip members and the protrusions.

13. The injection apparatus of claim 1, the base member comprising a connecting tube around which the support member is disposed, wherein the needle is fluidly coupled to the base member via the connecting tube.

14. An injection apparatus, comprising:
a hand-held unit having a head portion and a body portion, the head portion comprising
a base member comprising a connecting tube and an annular bore defined around the connecting tube,
a needle fluidly coupled to the connecting tube,
a safety cover coupled to the base member, the safety cover comprising one or more extension members extending past a distal end of the needle,
a movable portion disposed over the needle and being axially movable relative to the base member between an extended position and a retracted position in which a distal point of the needle is exposed,
a support member disposed within the annular bore of the base member,
an adjustment member rotatably coupled to the support member and releasably coupled to the movable portion such that rotation of the adjustment member adjusts a penetration depth of the needle by increasing or decreasing a distance between the movable portion and the base member.

15. The injection apparatus of claim 14, wherein the support member further comprises one or more resilient latches engaging a ridged portion disposed on an inner surface of the adjustment member.

16. The injection apparatus of claim 15, wherein the engagement of the latches with the ridged portion restrains the adjustment member against rotation relative to the support member unless a rotational force above a selected threshold is applied to the adjustment member.

17. The injection apparatus of claim 14, further comprising at least one control unit comprising a pump, the control unit being remote from the hand-held unit and fluidly coupled to the hand-held unit via one or more connecting tubes.

18. The injection apparatus of claim 14, wherein the hand-held unit is configured to be submersible in water.

19. The injection apparatus of claim 14, further comprising a biasing member disposed between the base member and the support member to bias the movable portion into the extended position.

20. The injection apparatus of claim 14, wherein the movable portion comprises one or more apertures through which the one or more extension members of the safety cover extend.

21. The injection apparatus of claim 14, further comprising a rod extending from the head portion into the body portion, the rod being configured to engage a sensor disposed within the body portion to trigger an injection of medicament when the movable portion is in the retracted position.

22. A method, comprising:
providing an injection apparatus according to claim 1; and
injecting a subject with at least one vaccine or medicament using the injection apparatus.

23. The method according to claim 22, further comprising:
disposing a subject between one or more extension members of a head portion of an injection apparatus, the head portion comprising a base member, a needle fluidly coupled to the base member, a movable portion disposed over the needle and being axially movable relative to the base member between an extended position and a retracted position in which a distal point of the needle is exposed, a safety cover removably coupled to the base member, the safety cover comprising the one or more extension members, and an adjustment member removably coupled to the movable portion and configured to adjust a penetration depth of the needle by increasing or decreasing a distance between the movable portion and the base member;
urging a selected implantation site of the subject against the movable portion thereby moving the movable portion from the extended position to the retracted position to expose the distal point of the needle; and
administering a medicament when the distal point of the needle has reached a selected depth within the subject.

24. A method, comprising;
providing components of an injection device comprising a hand-held unit having a head portion and a body portion, the head portion comprising a base member, a needle fluidly coupled to the base member, a movable portion disposed over the needle and being axially movable relative to the base member between an extended position and a retracted position in which a distal point of the needle is exposed, a safety cover removably coupled to the base member, the safety cover comprising one or more extension members, and an adjustment member removably coupled to the movable portion and configured to adjust a penetration depth of the needle by increasing or decreasing a distance between the movable portion and the base member; and
assembling the components to form an injection apparatus according to claim 1.
